(19) United States
(12) Patent Application Publication
(10) Pub. No.: US 2021/0374330 A1
(43) Pub. Date: Dec. 2, 2021

(54) INFORMATION INTEGRATION APPARATUS

(71) Applicants: OPExPARK INC., Tokyo (JP); TOKYO WOMEN'S MEDICAL UNIVERSITY, Tokyo (JP)

(72) Inventors: Go MUKUMOTO, Tokyo (JP); Hideki OKUDA, Aichi (JP); Yoshihiro MURAGAKI, Tokyo (JP); Jun OKAMOTO, Tokyo (JP)

(21) Appl. No.: 16/887,507

(22) Filed: May 29, 2020

(51) Int. Cl. G06F 40/169; G06F 40/117; G06F 16/23; G16H 30/40

(52) U.S. Cl. CPC G06F 40/169; G16H 30/40; G06F 16/2322; G06F 40/117

(57) ABSTRACT

The present disclosure provides a technique to easily share and record comments from users on information acquired from medical devices and displayed on a screen. An information integration apparatus of the present disclosure includes an information storage, a main image generator, a target designator, a comment inputter, an information generator, and a comment image generator.

### BACKGROUND

The present disclosure relates to a technique to display medical information acquired from a medical device used for surgery or treatment.

Japanese Unexamined Patent Application Publication No. 2015-185125 discloses a medical information system that acquires medical information from medical devices used for surgery and treatment, and integrates pieces of the medical information to display the integrated medical information on a screen.

### SUMMARY

This type of system is used for various purposes, including making a real-time consideration and providing real-time assistance during surgery, an after-surgery analysis, a review of, for example, a conference, an accident investigation, and an educational purpose. There have been demands for easy sharing of various information among those who are involved in such occasions. Information expected to be shared includes various information that is necessary for educational purposes and for preparing reference materials for conference presentations, such as details of decisions made during surgery, details of assistance provided during surgery, comments on setting changes for medical devices, comments on changes of measured values, things that those who are involved in surgery or treatment have noticed during the surgery or after the surgery.

It is desirable that one aspect of the present disclosure provides a technique to easily share and record comments from users on information acquired from medical devices and displayed on a screen.

One aspect of the present disclosure provides an information integration apparatus comprising an information storage, a main image generator, a target designator, a comment inputter, an information generator, and a comment image generator.

The information storage is configured to store pieces of device information respectively acquired from medical devices used for treating a patient. Each piece of the device information is stored in association with a corresponding first tag information. Each of the first tag information includes a time of acquisition of a corresponding piece of the device information, and a type of the corresponding piece of the device information.

The main image generator is configured to extract, among from the pieces of the device information stored in the information storage, pieces of the device information of at least one type each having the corresponding first tag information indicating an identical time, and to generate a main image data for displaying the pieces of the device information extracted on a single screen.

The target designator is configured to designate comment targets in an on-screen image displayed based on the main image data. The comment inputter is configured to input pieces of comment information associated with the respective comment targets designated by the target designator.

The information generator is configured to associate each piece of the comment information inputted from the comment inputter with a corresponding second tag information including an identification information for identifying a corresponding one of the comment targets designated by the target designator, and to store, in the information storage, the pieces of the comment information associated.

The comment image generator is configured, if the main image displayed based on the main image data includes at least one of the comment targets, to extract a corresponding piece of the comment information on at least one of the comment targets from the information storage, and to generate a comment image data for displaying the corresponding piece of the comment information extracted such that the corresponding piece of the comment information extracted is associated with the at least one of the comment targets in the main image.

The above-described configuration makes it possible to easily keep a record of, for example, a type of information that cannot be read from the device information as the comment information. Moreover, the comment information is displayed in association with the corresponding device information, which in turn enables a viewer of the display screen to clearly know what the target of the comment is.

### BRIEF DESCRIPTION OF THE DRAWINGS

Example embodiments of the present disclosure will be described hereinafter by way of example with reference to the accompanying drawings, in which:

FIG. 1 is a block diagram showing a configuration of an information integration apparatus;

FIG. 2 is a functional block diagram showing functions of the information integration apparatus;

FIG. 3 is an explanatory diagram showing a structure of tagged device information;

FIG. 4 is an explanatory diagram showing a structure of tagged comment information;

FIG. 5 is a flowchart illustrating a device information generation process;

FIG. 6 is a flowchart illustrating a display process;

FIG. 7 is an explanatory diagram showing information that is read out in the display process;

FIG. 8 is an explanatory diagram showing one example of a layout of a main image;

FIG. 9 is an explanatory diagram showing an event list;

FIG. 10 is a flowchart illustrating an add-comment process; and

FIG. 11 is an explanatory diagram showing a display example of the comment information.

### DETAILED DESCRIPTION OF EXEMPLARY EMBODIMENTS

### 1. Overall Configuration

An information integration apparatus 1 integrates various types of information available in an operation room, saves the integrated information, and displays the integrated information. The information integration apparatus 1 is used for, for example, making a real-time consideration and providing real-time assistance during surgery, for an after-surgery analysis, and for an educational purpose.

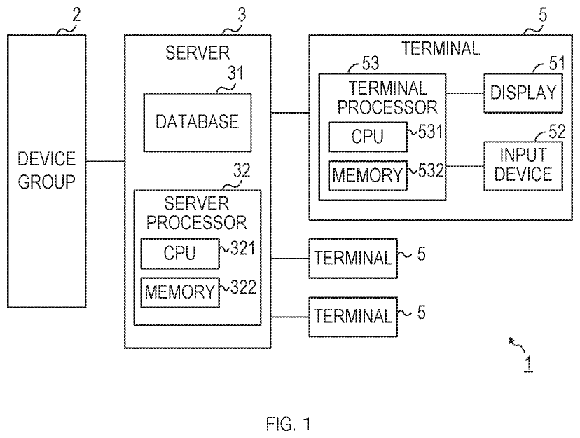

As shown in FIG. 1, the information integration apparatus 1 comprises a device group 2, a server 3, and terminals 5.

### 2. Device Group

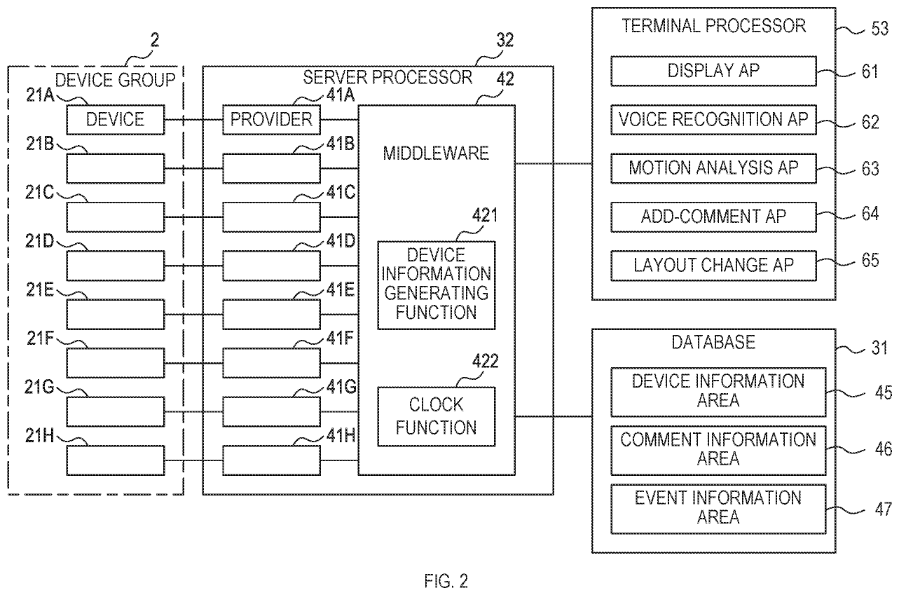

As shown in FIG. 2, the device group 2 comprises devices 21A to 21H. Each of the devices 21A to 21H generates data and feeds the data to the server 3. To refer to one of the devices 21A to 21H without specifically identifying which one of the devices 21A to 21H, the device is hereinafter mentioned as a device 21.

The device 21 is a medical system, a medical facility, and so on used in surgery and treatment. Specifically, examples of the medical system include a biological information monitor, a respiratory function monitor, a circulatory dynamics monitor, a sedation monitor, an anesthesia apparatus, an infusion pump, a syringe, a blood purification device, a heart-lung machine, and a ventricular assist device. Examples of the medical facility include a surgical navigation system (hereinafter, surgical navigator), an Internet Protocol (IP) camera, a medical gas system, an endoscope, a microscope, an electric scalpel, a drill, and an ultrasound irradiator. Examples of the device 21 may also include an air conditioning system and an isolated power system.

Examples of the data fed from the device 21 (hereinafter, device information) include measured values obtained by the device 21, moving images and still images outputted from the device 21, a device status of the device 21 such as a usage status and errors, set values of the device 21, and set values for parameters that the device 21 handles.

The surgical navigator acquires three-dimensional positions and postures of a surgical instrument during surgery. Examples of the surgical instrument include the microscope in addition to the electric scalpel and forceps. As a method for acquiring the three-dimensional positions and the postures of the surgical instrument, it is conceivable that the method comprises, for example, attaching markers in advance to specified positions of the surgical instrument, determining vectors from reference positions defined in advance in a space where the surgery is conducted to a given position of the surgical instrument to identify the three-dimensional position of the surgical instrument, and identifying the posture of the surgical instrument from a positional relationship between the markers. A focus of the microscope may be identified as the three-dimensional position. In this case, the focus of the microscope is associated with a three-dimensional image of an affected area of a patient captured in advance by, for example, Computed Tomography (CT) or Magnetic Resonance Imaging (MRI) to identify the three-dimensional position.

### 3. Server

The server 3 comprises a database 31 and a server processor 32.

The database 31 is realized by, for example, a hard disk drive (HDD), and includes, as shown in FIG. 2, at least a device information area 45, a comment information area 46, and an event information area 47. Tagged device information T_Idv is stored in the device information area 45, tagged comment information T_Icm is stored in the comment information area 46, and an event list L is stored in the event information area 47. The tagged device information T_Idv and the tagged comment information T_Iem are also collectively referred to as integrated information. The server 3 corresponds to one example of the information storage.

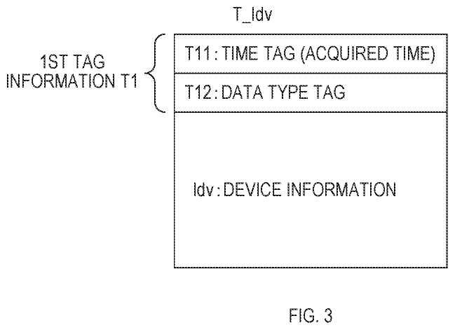

As shown in FIG. 3, the tagged device information T_Idv comprises first tag information T1 and the device information Idv.

The device information Idv is acquired from the device 21 of the device group 2 during surgery, for example, and specifically includes measured values, set values, and image data.

The first tag information T1 is information related to the device information Idv and includes at least a time tag T11 and a data type tag T12. The time tag T11 indicates time when the device information Idv was acquired (hereinafter, acquired time). The data type tag T12 includes identification information for identifying the device 21 in which the device information Idv is generated. In a case where two or more pieces of the device information Idv are acquired from one device 21, each of the data type tags T12 also includes identification information for distinguishing the device information Idv from other pieces of the device information Idv.

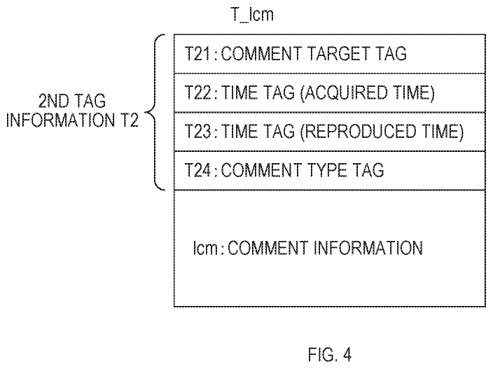

As shown in FIG. 4, the tagged comment information T_Icm comprises second tag information T2 and comment information Icm.

The comment information Icm is inputted through the terminal 5 during surgery or after the surgery, and specifically includes text data, audio data, and image data. The image data may include bitmap data of hand-written letters and of manual drawing.

The second tag information T2 is information related to the comment information Icm and includes at least a comment target tag T21, a time tag T22, a time tag T23, and a comment type tag T24.

The comment target tag T21 is information for identifying a comment target. Examples of the comment target include an entirety or a portion of the device information Idv identified by the data type tag T12, and an entirety of information in which no device information Idv is identified (that is, an entire screen in which pieces of the device information Idv are displayed). In a case where the device information Idv is a measured value obtained by the device 21 and the measured value is displayed in the form of a numbers or a graph, the “portion of the device information Idv” includes the displayed numbers or the graph.

The time tag T22 indicates the acquired time of the comment target. That is, the time tag T22 indicates the same time as the time indicated by the above-described time tag T11. In other words, the tagged comment information T_Icm is associated with the tagged device information T_Idv because of the comment target tag T21 and the time tag T22.

The time tag T23 indicates time when the comment target is designated when the comment information Icm is inputted. The time tag T23 indicates current time when the integrated information is being reproduced (hereinafter, reproduced time). In a case where the integrated information is reproduced in real time, that is, in a case where the integrated information is concurrently generated and reproduced, the value of the time tag T23 may be the same as that of the time tag T22.

The comment type tag T24 is information indicating the form of the comment information Icm, and the example thereof includes text data, audio data, image data, voice recognition data, and motion analysis data.

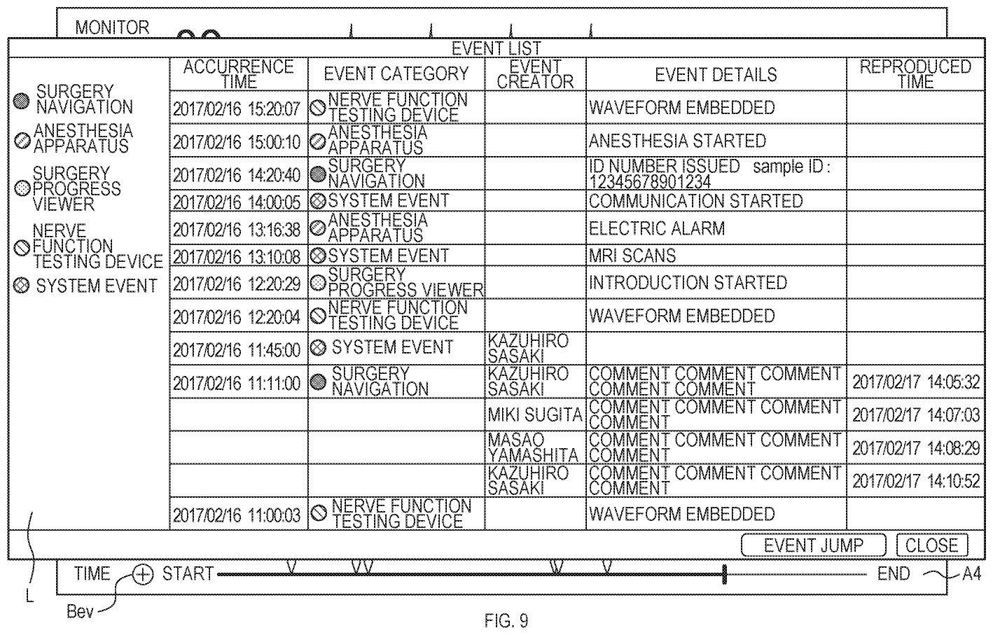

As shown in FIG. 9, the event list L includes at least “occurrence time”, “event category”, “event creator”, “event description”, and “reproduced time” as items of information. The “occurrence time” is time when an event takes place, and indicates a time point on the same time-axis as that of the acquired time. The “event category” shows the category of the occurred event, and specifically indicates which device 21 is involved in the event. The “event creator” indicates who has operated the terminal 5 to create the event, if the event is created by an input from the terminal 5.

The “event creator” may be inputted through the terminal 5, or information on a user of the terminal 5 registered in advance may be acquired from the terminal 5. The “event description” shows details of the occurred event, and shows the contents of an inputted comment, if the event is an input of a comment event, which will be described later. The “reproduced time” is time when an event is registered, in a case where the event is additionally registered on the event list L while the integrated information is reproduced, and indicates the current time when the integrated information is being reproduced.

The cells for the “event creator” and the “event description” are suitably filled in through the terminal 5.

Referring back to FIG. 1, the server processor 32 comprises a microcomputer including a CPU 321 and semiconductor memories, such as a RAM and a ROM (hereinafter, memory 322).

As shown in FIG. 2, the server processor 32 functionally comprises providers 41A to 41H and a middleware 42. To refer to one of the providers 41A to 41H without specifically identifying which one of the providers 41A to 41H, the provider will be hereinafter mentioned as a provider 41.

To each of the providers 41A to 41H, one of the devices 21A to 21H is connected. The provider 41 transmits information to and receives information from the connected device 21.

The middleware 42 is a communication interface that provides, together with the providers 41A to 41H, unified access means to various types of the devices 21A to 21H connected to the corresponding providers 41A to 41H irrespective of differences of programming languages and communication protocols. In the present embodiment, an Open Resource Interface for Network (ORiN) is used as the middleware 42.

The middleware 42 comprises at least a device information generating function 421 to generate the tagged device information T_Idv, and a clock function 422 to provide the current time upon request.

### 3-1. Device Information Processing

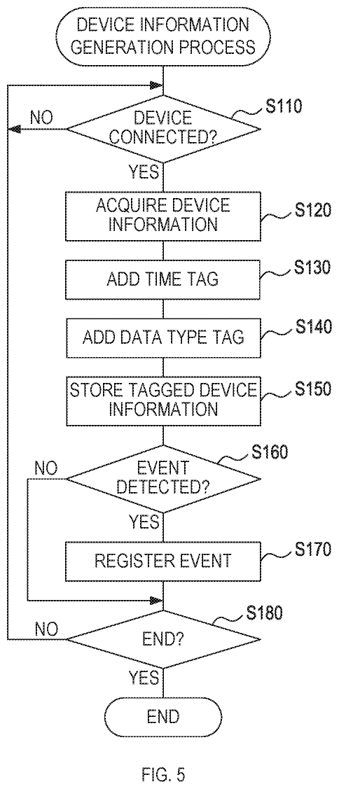

Referring to the flowchart of FIG. 5, the following describes processing (hereinafter, device information processing) performed to implement the device information generating function 421 of the middleware 42 of the server processor 32. The present processing is performed in each of the providers 41A to 41H when the server 3 is turned on.

In S110, the server processor 32 determines whether the device 21 is connected to the provider 41 in which the present processing is performed. If the server processor 32 determines that the device 21 is not connected, the server processor 32 repeats the same step while waiting for the device 21 to be connected. If the server processor 32 determines that the device 21 is connected, the process proceeds to S120.

In S120, the server processor 32 acquires the device information Idv from the device 21 via the provider 41. The device information Idv acquired from one device 21 may be one type of device information or two or more types of device information.

Subsequently in S130, the server processor 32 acquires the current time to generate the time tag T11, and adds the time tag T11 to the device information Idv acquired in S110. The current time may be acquired using the clock function 422 of the microcomputer, or time information indicating the acquired time of the device information Idv, which is provided from the device 21 together with the device information Idv, may be used.

Subsequently in S140, the server processor 32 adds the data type tag T12, indicating the type of the device information Idv, to the device information Idv acquired in S120. Among the data type tags T12 prepared in advance, a suitable data type tag T12 is used depending on the device 21 connected to the provider 41.

Subsequently in S150, the server processor 32 stores the tagged device information T_Idv, in which the first tag information T1 including the time tag T11 and the data type tag T12 is added to the device information Idv in the above-described processing, in the device information area 45 of the database 31.

Subsequently in S160, the server processor 32 determines whether any event has occurred in relation to the device information Idv. An event occurs when the device information Idv satisfies a given event condition. The event condition may be, for example, in a case where the device information Idv is a measured value, the measured value, or a differential value or an integrated value of the measured value exceeding a specified threshold. However, the event condition is not limited to this.

If the server processor 32 determines that an event has occurred, the process proceeds to S170. If the server processor 32 determines that no event has occurred, the process proceeds to S180.

In S170, the server processor 32 registers event occurrence information, indicating the time when the event has occurred, and event target information, indicating the device information Idv in relation to which the event has occurred, in the event list L stored in the event information area 47. Then, the process proceeds to S180.

In S180, the server processor 32 determines whether to terminate generation of the device information Idv. Specifically, the server processor 32 determines whether a command to terminate the processing has been inputted via the terminals 5. If the server processor 32 determines to continue to generate the device information Idv, the process returns to S110. If the server processor 32 determines to terminate generation of the device information Idv, the processing is terminated.

### 4. Terminals

As shown in FIG. 1, each of the terminals 5 comprises a display 51, an input device 52, and a terminal processor 53. All the terminals 5 have the same configurations.

The display 51 is, for example, a touchscreen-type liquid crystal display, and can show three-dimensional images such as Multi-Planar Reconstruction (MPR) images, waveform data and values indicating results of measurements, such biological information, various operation buttons, set values for each piece of equipment, and display setting values.

The input device 52 is, for example, input equipment such as the touchscreen provided to the above-described liquid crystal display, a keyboard, a mouse, a microphone, a camera, and a motion sensor. The input device 52 outputs, for example, text data, position data, audio data, and image data, corresponding to input operations through the above-mentioned input equipment, to the terminal processor 53.

The terminal processor 53 comprises a microcomputer including a CPU 531 and semiconductor memories, such as a RAM and a ROM (hereinafter, memory 532). The terminal processor 53 executes one or more application(s) (hereinafter, AP) in relation to the information stored in the database 31 of the server 3. As shown in FIG. 2, the terminal processor 53 executes at least a display AP 61, a voice recognition AP 62, a motion analysis AP 63, an add-comment AP 64, and a layout change AP 65 in the present embodiment.

The voice recognition AP 62 is designed for performing a voice recognition process with respect to user's voice received with the microphone, which is one of the input equipment of the input device 52, to generate the voice recognition data that is in the form of a command or text data depending on the recognition result, and to output the voice recognition data in combination with the voice that has undergone the voice recognition process.

The motion analysis AP 63 is designed for analyzing images of motion of the user (for example, movement of hand(s), face, head, and so on) captured by the camera, which is one of the input equipment of the input device 52, to generate the motion analysis data that is in the form of a command or text data depending on the analysis result, and to output the motion analysis data in combination with the images that have undergone the analysis.

In other words, the input device 52, the voice recognition AP 62, and the motion analysis AP 63 of the terminal 5 enables a non-contact input operation.

### 4-1. Display Process

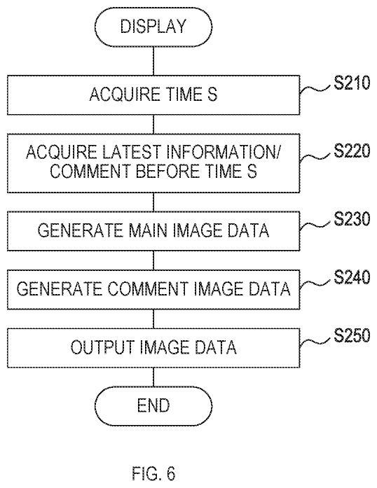

Referring to the flowchart in FIG. 6, the following describes a process (hereinafter, display process) performed by the terminal processor 53 executing the display AP 61.

The display process is repeated in a given display cycle during a period from when a start display command is inputted through the input device 52 until when a stop display command is inputted. In the display process, the device information Idv and the comment information Icm corresponding to time S designated through the input device 52 are shown on a display screen of the display 51 based on the integrated information stored in the database 31. In an operation mode in which still images are displayed, the time S is changed in accordance with an input from the input device 52, whereas, in an operation mode in which moving images are displayed, the time S is automatically updated in synchronization with the display cycle after an initial value is inputted from the input device 52.

In S210, the terminal processor 53 acquires the time S.

Subsequently in S220, the terminal processor 53 acquires, for each device type and each comment type, the device information Idv with the time tag T11 and the comment information Icm with the time tag T22, in which the time tag T11 and the time tag T12 indicate the latest time points before the time S, from the database 31. In a search for the latest information, a search limit up to which the search can go back may be defined.

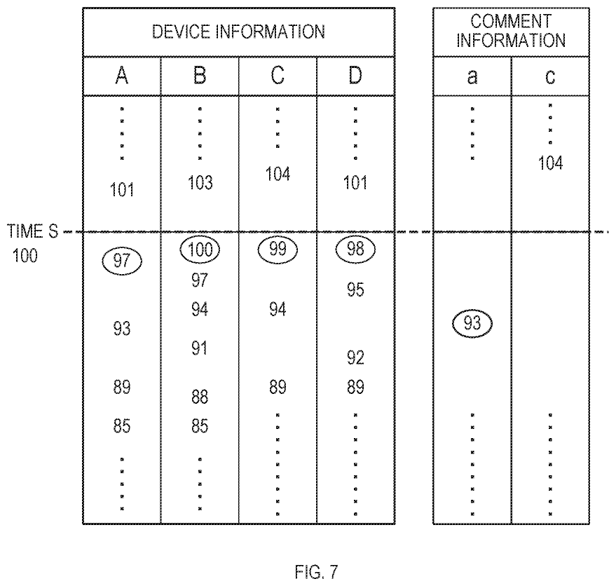

For example, as shown in FIG. 7, assume that the database 31 stores four types of the device information Idv identified as A to D, and two types of the comment information Icm identified as a and c. As shown in FIG. 7, in a case where the time S is X millisecond past 9:00, the hour unit and the minute unit are omitted, and the millisecond unit “X” is shown. In other words, the row of numbers in column A in FIG. 7 show the contents of the time tag T11 added to the device information Idv of a device type A.

In a case where the time S is at “100”, pieces of the device information Idv with the latest time tag T11 before the time S are, as shown with circles in FIG. 7: the device information Idv with the time tag T11 indicating “97” for the device type A; the device information Idv with the time tag T11 indicating “100” for a device type B; the device information Idv with the time tag T11 indicating “99” for a device type C; and the device information Idv with the time tag T11 indicating “98” for a device type D.

The comment information Icm with the latest time tag T22 before the time S for the device type a is the comment information with the time tag T22 indicating “93”. FIG. 7 shows that there is no applicable comment information Icm for the device type c within a search range defined by the search limit.

Subsequently in S230, the terminal processor 53 generates main image data for displaying the device information Idv read in S220 in conformity with a layout set at that time. Hereinafter, an image displayed based on the main image data is referred to as a main image.

The above-mentioned layout is a combination of the following elements: (1) the way to divide a display area of the display screen; (2) the types of the device information Idv and the number of pieces of the device information Idv to be concurrently displayed in each of the divided areas; and (3) the form (an image, a numerical value, a graph, and so on) of the device information Idv to be displayed in each of the divided areas.

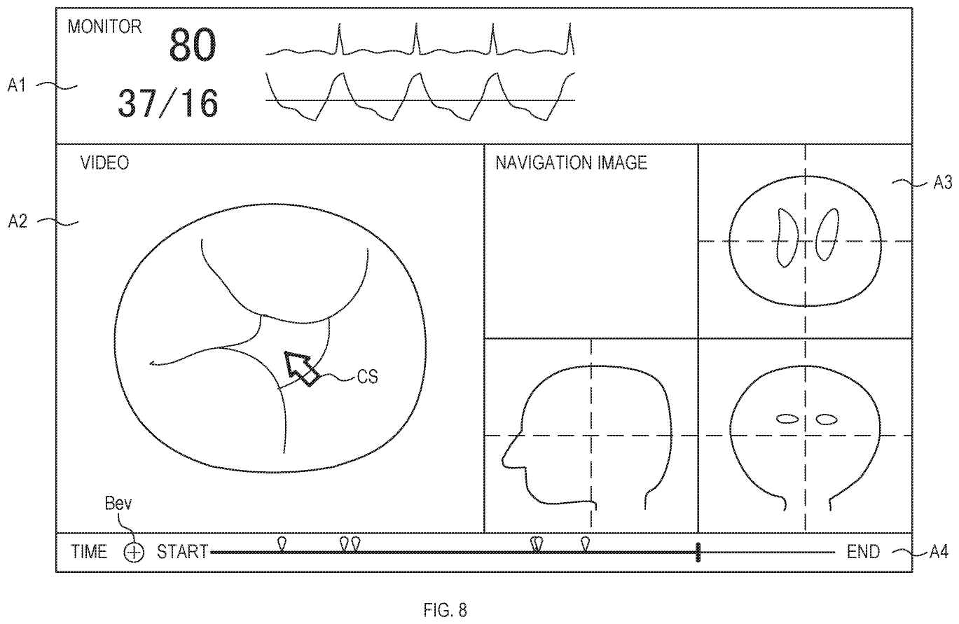

As shown in FIG. 8, one example of the layout of the main image may include a monitor area A1, a video area A2, a navigation image area A3, and a time area A4. The monitor area A1 shows graphs, for example, for monitoring the biological information. In the monitor area A1, the biological information is displayed in the forms of numerical values and graphs.

The video area A2 shows moving images of a treatment target captured by the camera, for example. The navigation image area A3 shows the three-dimensional position detected by the surgical navigator in combination with three-dimensional images of the affected area prepared in advance. In the navigation image area A3, three cross-sectional images (hereinafter, navigation images) are displayed in a triple-view manner each showing the detected three-dimensional position in a cross-section different from each other.

The time area A4 shows a time range, in which a chronological sequence of the integrated information exists, is expressed in the form of a time axis, and shows the time S on the time axis. In the time area A4, balloon-like indicators may be shown along with the time axis to show the occurrence time of events. Setting and changing of the layout are implemented by execution of the layout change AP 65, which is separately provided.

Referring back to FIG. 6, subsequently in S240, the terminal processor 53 generates comment image data for displaying the comment information Icm read in S220 in combination with the main image. Hereinafter, an image displayed based on the comment image data is referred to as a comment image. The display position and the display format of the comment image in the main image are specified in advance in accordance with the comment type of the comment information Icm and the data type of the device information Idv designated as the comment target. There may be an application specifically for changing the display position and the display format of the comment image.

Subsequently in S250, the terminal processor 53 outputs the main image data generated in S230 and the comment image data generated in S240 to the display 51 so that the main image and the comment image are shown on the display screen of the display 51. Then the process temporarily ends.

S210 to S230 correspond to one example of the main image generator, and S210 to S220, and S240 correspond to one example of the comment image generator.

### 4-2. Add-Comment Process

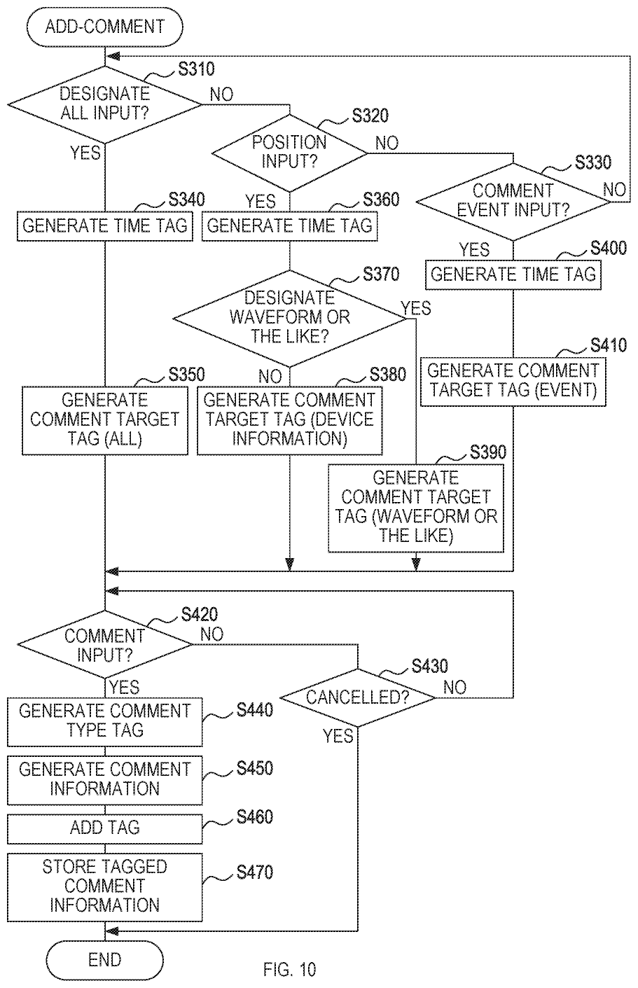

Referring to the flowchart in FIG. 10, the following describes a process (hereinafter, add-comment process) performed by the terminal processor 53 executing the display AP 61. The present process is repeated while the display process is performed.

In S310, the terminal processor 53 determines whether a designate-all input has been entered through the input device 52 to designate all the information (hereinafter “all”) shown on the screen of the display 51 (hereinafter, display screen) as the comment target. If the terminal processor 53 determines that the designate-all input has been entered, the process proceeds to S340. If the terminal processor 53 determines that the designate-all input has not been entered, the process proceeds to S320.

In S340, the terminal processor 53 acquires the acquired time, corresponding to a timing at which the designate-all input is entered, to generate the time tag T22, and acquires the reproduced time, corresponding to the aforementioned timing, to generate the time tag T23. The acquired time mentioned here is the time S most recently acquired in S210 of the display process performed before the aforementioned timing, or the time indicated by the time tag T11 associated with the device information Idv that is being displayed at the aforementioned timing. The reproduced time mentioned here is the current time acquired from the clock function 422 at the aforementioned timing.

Subsequently in S350, the terminal processor 53 generates the comment target tag T21 indicating that the comment target is “all”, and then the process proceeds to S420.

In S320, the terminal processor 53 determines whether a position input, indicating a position on the display screen, has been entered through the input device 52. The position indicated by the position input may be a position inputted via the touchscreen of the display screen, or a position of a cursor on the display screen that is shown by operation of the mouse or the keyboard.

Moreover, in a case where the navigation image area A3 is included in the main image, the position may be the three-dimensional position shown in the navigation images. Specifically, the three-dimensional position is located in the center of each of the navigation images at which auxiliary lines intersect, which is shown in each of the triple views. If the terminal processor 53 determines that the position input has been entered, the process proceeds to S360. If the terminal processor 53 determines that the position input has not been entered, the process proceeds to S330.

In S360, the terminal processor 53 acquires the acquired time and the reproduced time corresponding to a timing at which the position input is entered through the input device 52, and generates the time tags T22 and T23 in the same manner as in S340.

Subsequently in S370, the terminal processor 53 determines whether the numbers indicating the measured value or a waveform (hereinafter, waveform or the like) are/is shown in a designated position on the display screen specified through the position input. That is, the terminal processor 53 determines whether the waveform or the like is designated through the position input. If the terminal processor 53 determines that the waveform or the like is not designated, the process proceeds to S380. If the terminal processor 53 determines that the waveform or the like is designated, the process proceeds to S390.

In S380, the terminal processor 53 identifies the designated position and the type of the device information Idv displayed in the designated position based on the layout of the main image at that time. The type of the device information Idv is indicated by the data type tag T12 associated with the device information Idv. Moreover, the terminal processor 53 generates the comment target tag T21 indicating that the identified device information Idv is the comment target, and then process proceeds to S420.

In S390, the terminal processor 53 generates the comment target tag T21 indicating that the waveform or the like designated by the position input is the comment target, and then process proceeds to S420.

In S380 and S390, the comment target tag T21 may include information indicating the designated position using information on relative positions of the components of the comment target in the display area allocated to the comment target. This information is to inhibit the relation between the displayed components in the display area and the designated position from being changed when the layout is changed.

In S330, the terminal processor 53 determines whether a comment event input, which is an operation to input a comment through the input device 52, has been entered. Specifically, the terminal processor 53 determines that the comment event input has been entered, when an add-event button Bev displayed in the time area A4 of the display screen as shown in FIG. 8 is operated through the input device 52. If the terminal processor 53 determines that the comment event input has been entered, the process proceeds to S400. If the terminal processor 53 determines that the comment event input has not been entered, the process return to S310. When the add-event button Bev is operated, the event list L, in which a new comment event has been added, is displayed as shown in FIG. 9.

In S400, the terminal processor 53 acquires the acquired time and the reproduced time corresponding to a timing at which the comment event input is entered, and generates the time tags T22 and T23 in the same manner as in S340. The acquired time and the reproduced time are also shown in the event list L.

Subsequently in S410, the terminal processor 53 generates the comment target tag T21 indicating that the comment target is “event”, and then the process proceeds to S420.

In S420, the terminal processor 53 determines whether a comment has been entered through the input device 52. If the terminal processor 53 determines that a comment has been inputted, the process proceeds to S440. If the terminal processor 53 determines that a comment has not been inputted, the process proceeds to S430.

In S430, the terminal processor 53 determines whether a command to cancel the comment input has been entered through the input device 52 (hereinafter, cancelation command). If the terminal processor 53 determines that the cancelation command has been entered, the terminal processor 53 ends the process. If the terminal processor 53 determines that the cancelation command has not been entered, the process returns to S420.

In S440, the terminal processor 53 identifies the type of the comment inputted from the input equipment used when the comment is inputted, and generates the comment type tag T24. Examples of the comment type include a text input through the keyboard, a handwriting input through the touchscreen, an audio input through the microphone, and an image input through the camera.

Subsequently in S450, the terminal processor 53 generates the comment information Icm in accordance with the comment type. Specifically, if the comment type is the text input or the handwriting input, the inputted text data or the inputted image is used as it is as the comment information Icm. If the comment type is the audio input, in addition to the audio data, the voice recognition data generated by execution of the voice recognition AP 62 is also included in the comment information Icm. Similarly, if the comment type is the image input, in addition to the image data, the motion analysis data generated by execution of the motion analysis AP 63 is also included in the comment information Icm.

Subsequently in S460, the terminal processor 53 adds the time tags T22 and T23 generated in S340, S360, and S400, the comment target tags T21 generated in S350, S380, S390, and S410, and the comment type tag T24 generated in S440 to the comment information Icm generated in S450 to generate the tagged comment information T_Icm.

Subsequently in S470, the terminal processor 53 stores the generated tagged comment information T_Icm in the database 31, and then temporarily ends the process.

The contents of the tagged comment information T_Icm stored in the database 31 are immediately reflected and displayed by the repeatedly performed display process.

S310 to S330 and the input device 52 correspond to one example of the target designator. S420 and the input device 52 corresponds to one example of the comment inputter. S340 to S410 and S440 to S470 correspond to one example of the information generator.

Each function of the server processor 32 and that of the terminal processor 53 are respectively implemented by the CPU 321 and the CPU 531 executing the programs stored in non-transitory tangible recording media. In the present embodiment, the memories 322 and 532 correspond to one example of the non-transitory tangible recording media in which the programs are stored. Moreover, when these programs are executed, the methods corresponding to the programs are performed. The server processor 32 and the terminal processor 53 may each comprise one microcomputer, or two or more microcomputers.

A way to implement the function(s) of each component of the server processor 32 and that of the terminal processor 53 is not limited to running software. The function may be partly or entirely implemented by use of one or more types of hardware. For example, in a case where the aforementioned function is implemented by an electronic circuit, which is hardware, the electronic circuit may be realized by a digital circuit, an analog circuit, or a combination of these circuits.

### 5. Display Examples

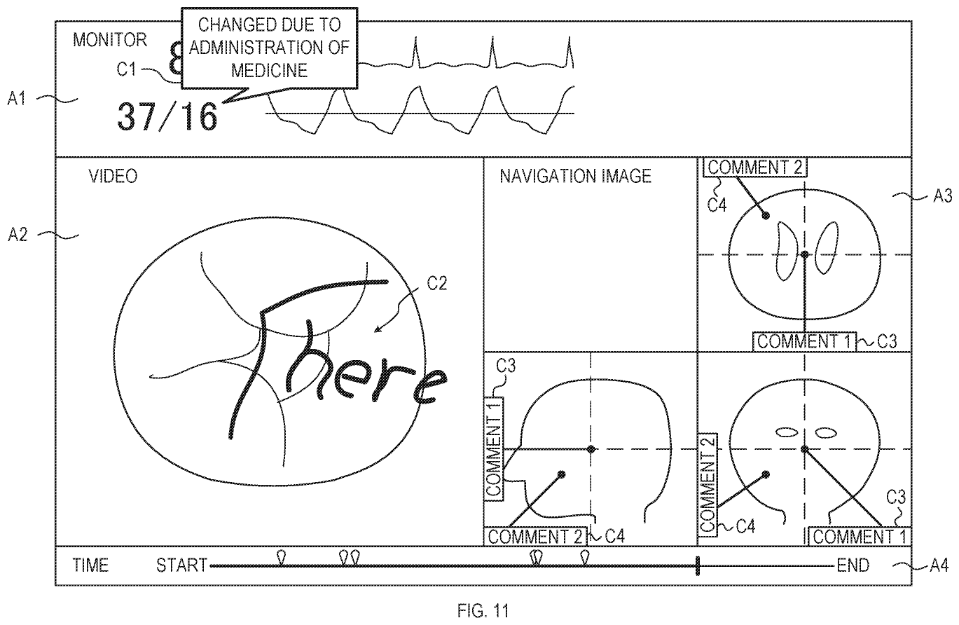

With reference to FIG. 11, the following describes display examples of the comment information Icm as a result of the display process.

In a case where the measured value displayed in the monitor area A1 significantly changes, the position input is entered by, for example, finger touch on the portion of the screen in which the measured value is shown, and then a comment such as “changed due to administration of medicine” is inputted through, for example, the keyboard. Accordingly, as shown by C1 in FIG. 11, a text comment related to the parameter designated as the comment target is displayed.

In a case where the position input is entered to designate the video area A2, a line drawing is then manually drawn and text is handwritten on the display screen using an input pen for the touchscreen. Accordingly, as indicated by a reference numeral C2 in FIG. 11, the manually-drawn line drawing and the handwritten text are displayed in their original forms overlapping with the image in the video area A2 that is designated as the comment target.

In a case where an operation is performed to designate the navigation image area A3 while the MPR image is displayed in the triple-view manner in the navigation image area A3, it is determined that the position input has been entered to designate the three-dimensional position identified by the MPR image displayed in the navigation image area A3. Thus, it becomes possible to input a comment on the designated three-dimensional position. In this case, three-dimensional position shown in the center of each of the triple views becomes the comment target, and the comment thereon is displayed in the portion “Comment 1” indicated by a reference numeral C3 in FIG. 11.

In another position in each of the triple views, a comment on a black dot indicating a three-dimensional position previously designated as the comment target is displayed in the portion “Comment 2” indicated by a reference numeral C4 in FIG. 11. The comments may be shown in a manner not to overlap with the images, and may be shown in space in the navigation image area A3 where there is no image.

Although it is not depicted, in a case where the designate-all input is entered, a vertically elongated comment display area may be provided, for example, in the upper right corner of the display screen. In the comment display area, time of comment entry and contents of comments may be chronologically displayed.

### 6. Effects

The information integration apparatus 1 described above achieves the following effects.

(6a) In a case where the device information Idv is displayed in real time during surgery or treatment, the status of the surgery or the treatment, for example, can be shared in real time with anyone who are involved in the surgery or the treatment. In addition, it is possible to leave real time comments as memorandums of, for example, advices given during surgery, explanation of the intention of operating the device(s) or the intention of the work performed during the surgery, and comments on things that those who are involved in the surgery or the treatment have noticed during the surgery.

For example, in a case where the device information Idv shown on the display screen includes information indicating a distinctive phenomenon, feature, and so on, it is possible to leave comments on the information that cannot be read from the device information Idv, such as administration of medicine, a change of setting of the air conditioner, and a change of posture of the patient.

(6b) In addition to an input of comments through the operating equipment such as the touchscreen, the keyboard, and the mouse, a non-contact input of comments is also possible by voice through the microphone and gestures through the camera. Accordingly, a comment from, for example, a practitioner who is under circumstances where the practitioner cannot operate the equipment can also be easily left.

(6c) In a case where the device information Idv and the comment information Icm are displayed after surgery or treatment for a feedback of the surgery or the treatment, for an educational purpose, and so on, a viewer can acquire, from the comment information Icm, useful information that cannot be achieved from the device information Idv alone. It is also possible to add a new comment during the after-surgery viewing.

(6d) When an comment is inputted, each piece of the device information Idv shown on the display screen can be designated as the comment target, and the comment information Icm is shown or reproduced in association with the designated device information Idv. Accordingly, the viewer can clearly know what the comment target is.

(6e) Since “all” can be designated as the comment target, viewers can have discussion over the display screen and leave the contents of the discussion in the form of comments using this function.

### 7. Other Embodiments

The above has described an embodiment of the present disclosure. However, the present disclosure is not limited to the above-described embodiment and may be implemented in various ways.

(7a) In the aforementioned embodiment, the applications 61 to 65 are installed in the terminal 5. However, the present disclosure is not limited to this. For example, the terminal 5 may be integrated with the server 3, and the applications 61 to 65 may be at least partly installed in the server 3.

(7b) Functions of one component in the above-described embodiments may be achieved by two or more components, and a function of one component may be achieved by two or more components. Moreover, functions of two or more components may be achieved by one component, and a function achieved by two or more components may be achieved by one component. Furthermore, one part of the configurations of the above-described embodiments may be omitted. Moreover, at least a part of the configurations of the aforementioned embodiments may be added to or replaced with other configurations of the aforementioned embodiments.

(7c) In addition to the above-described information integration apparatus, the present disclosure can be implemented in various forms including a system comprising the information integration apparatus as a component, a program that makes a computer function as the information integration apparatus, and a non-transitory tangible recording medium, such as a semiconductor memory, that stores the program.

1. An information integration apparatus comprising:
an information storage configured to store pieces of device information respectively acquired from medical devices used for treating a patient, each piece of the device information being stored in association with a corresponding first tag information, each of the first tag information including:
a time of acquisition of a corresponding piece of the device information, and
a type of the corresponding piece of the device information;
a main image generator processor configured to extract, among from the pieces of the device information stored in the information storage, pieces of the device information of at least two types each having the corresponding first tag information indicating an identical time, and to generate a main image data for displaying the pieces of the device information extracted on a single screen;
a target designator processor configured to designate comment targets in an on-screen image displayed based on the main image data;
a comment inputter processor configured to input pieces of comment information associated with the respective comment targets designated by the target designator processor;
an information generator processor configured to associate each piece of the comment information inputted from the comment inputter processor with a corresponding second tag information including an identification information for identifying a corresponding one of the comment targets designated by the target designator processor, and to store, in the information storage, the pieces of the comment information associated; and
a comment image generator processor configured, if a main image displayed based on the main image data includes at least one of the comment targets, to extract a corresponding piece of the comment information on at least one of the comment targets from the information storage, and to generate a comment image data for displaying the corresponding piece of the comment information extracted such that the corresponding piece of the comment information extracted is associated with the at least one of the comment targets in the main image.

2. The information integration apparatus according to claim 1,
wherein the target designator processor is configured to designate a position on a screen displaying the on-screen image, and
wherein one of the comment targets includes an entirety or a part of the corresponding piece of the device information displayed in the position designated by the target designator processor.

3. The information integration apparatus according to claim 2,
wherein one piece of the device information includes a control parameter related to a corresponding one of the medical devices.

4. The information integration apparatus according to claim 2,
wherein one piece of the device information includes a graph showing a waveform of biological information.

5. The information integration apparatus according to claim 1,
wherein one piece of the device information includes a three-dimensional position of a surgical instrument during surgery, and
wherein one of the comment targets includes a navigation image of a surgical navigation system showing a three-dimensional image of a treatment target that matches the three-dimensional position of the surgical instrument.

6. The information integration apparatus according to claim 1,
wherein one of the comment targets includes an entirety of the on-screen image.

7. The information integration apparatus according to claim 6,
wherein the target designator processor is configured to designate the entirety of the on-screen image as one of the comment targets in response to an operation of an operating portion provided in advance.

8. The information integration apparatus according to claim 1,
wherein the device information includes at least one of: a measured value obtained by the device; a moving image outputted from the device; a still image outputted from the device; a usage status of the device; a device status of the device; a set value of the device; and a set value for parameters that the device handles.

9. The information integration apparatus according to claim 1,
wherein the main image generator processor is configured to generate the main image data in conformity with a preset layout.

10. An information integration apparatus comprising:
an information storage configured to store pieces of device information respectively acquired from medical devices used for treating a patient, each piece of the device information being stored in association with a corresponding first tag information, each of the first tag information including:
a time of acquisition of a corresponding piece of the device information, and
a type of the corresponding piece of the device information;
a main image generator processor configured to extract, among from the pieces of the device information stored in the information storage, pieces of the device information of at least two types each having the corresponding first tag information indicating an identical time, and to generate a main image data for displaying the pieces of the device information extracted on a single screen;
a target designator processor configured to designate comment targets in an on-screen image displayed based on the main image data;
a comment inputter processor configured to input pieces of comment information associated with the respective comment targets designated by the target designator processor;
an information generator processor configured to associate each piece of the comment information inputted from the comment inputter processor with a corresponding second tag information including an identification information for identifying a corresponding one of the comment targets designated by the target designator processor, and to store, in the information storage, the pieces of the comment information associated; and
a comment image generator processor configured, if a main image displayed based on the main image data includes at least one of the comment targets, to extract a corresponding piece of the comment information on at least one of the comment targets from the information storage, and to generate a comment image data for displaying the corresponding piece of the comment information extracted such that the corresponding piece of the comment information extracted is associated with the at least one of the comment targets in the main image,
wherein the main image generator processor is configured to generate the main image data in conformity with a way to divide a display area of a display screen into divided areas and in conformity with one of the following elements:
(1) types of the device information and a number of pieces of the device information to be concurrently displayed in each of the divided areas; and
(2) a form of the device information to be displayed in each of the divided areas.
